(19) United States
(12) Patent Application Publication
(10) Pub. No.: US 2011/0111432 A1
(43) Pub. Date: May 12, 2011

(54) DIAGNOSTIC METHOD FOR PEANUT ALLERGY

(76) Inventors: Manel Jordana, Hamilton (CA); Susan Waserman, Toronto (CA)

(21) Appl. No.: 12/922,453

(22) PCT Filed: Mar. 12, 2009
(86) PCT No.: PCT/CA2009/000321

§ 371 Date: Dec. 17, 2010

Related U.S. Application Data
(62) Division of application No. 61/064,571, filed on Mar. 12, 2008.

(51) Int. Cl. G01N 33/53; G06N 5/02

(52) U.S. Cl. 435/7.21; 435/7.92; 706/46

(57) ABSTRACT

The present invention provides novel methods and tools to differentiate between true allergy and false positive allergy tests. In particular, parameters that can be statistically associated with true peanut allergy have been identified. These include wheal size in response to a skin prick test and total IgE. Further parameters may be measured for greater certainty.

### CROSS-REFERENCE TO RELATED APPLICATIONS

This application is a Submission Under 35 U.S.C. §371 for U.S. National Stage Patent Application of International Application Number PCT/CA2009/000321, filed Mar. 12, 2009, and entitled DIAGNOSTIC METHOD FOR PEANUT ALLERGY, which is related to and claims priority to U.S. Patent Application Ser. No. 61/064,571, filed Mar. 12, 2008, the entirety of both are incorporated herein by reference.

### FIELD OF INVENTION

The present invention relates to methods and systems for diagnosing food allergies.

### BACKGROUND OF THE INVENTION

The prevalence of food allergies has been on the rise for many years and clinicians require accurate diagnostic tests to determine whether there is a true allergy. The public's perception of the number of individuals affected by food allergies appears to be in excess of what can actually be demonstrated clinically. As a means of possibly preventing the allergy, parents will start to withhold certain foods at a very young age.

Although an individual can be allergic to any food, 90% of all food-allergic reactions are to one of the following foods: milk, egg, peanut, tree nut (walnut, cashew, etc.), fish, shellfish, soy, and wheat.

Peanuts are one of the most allergenic foods. Sensitivity to peanuts may result in symptoms ranging from mild urticaria to severe systemic anaphylaxis. Peanuts are the food most commonly associated with fatal anaphylaxis.

Current diagnostic methods including skin prick tests (SPT) and blood tests (ImmunoCap) cannot always reliably predict who is peanut allergic. SPT, while very sensitive (greater than 90%) have poor specificity (50-60%). The ImmunoCap, a blood test which measures circulating peanut-specific IgE, may help assess the risk of an allergic reaction on oral challenge with peanut, but the combined specificity of SPT and ImmunoCap remains low at 60-70%. It has also been proposed that SPT wheal diameters greater than 8 mm are 100% predictive for peanut allergy, and that high peanut-specific IgE values (≧15 KU/L) have a 95% positive predictive value. Although these proposed 95% predictive precision points have some clinical utility, many patients have SPT or specific IgE values that fall into an intermediate, poorly predictive, range. Moreover, these measurements can vary over time related to peanut intake, symptoms, and age.

A particularly problematic group is children who have never been exposed to peanut, yet because of an atopic diathesis have a positive peanut skin test. The only means of definitively diagnosing this group is with an oral peanut challenge, a potentially risky procedure that can also yield equivocal results. Accurate diagnosis of peanut allergy is essential. There is significant impact on quality of life in adhering to a peanut-free diet which is the current treatment of choice.

The present invention addresses the need for alternative methods for the diagnosis of food allergies, particularly peanut allergy.

### SUMMARY OF THE INVENTION

The present invention provides methods and tools for the diagnosis of allergy, particularly food allergies, more particularly peanut allergy. A diagnostic algorithm is provided which includes a series of demographic and biological variables such as age, size of prick test response, level of total serum IgE and interferon gamma production by blood mononuclear cells. The variables are hierarchically organized to provide a decision tree dependent on a numerical value for each variable. Different paths outline different predictions for true allergy. Specific paths were found to predict true peanut allergy with very high sensitivity.

In one aspect of the invention a method for diagnosing an allergy in a subject is provided. The method comprises: a) measuring at least one variable selected from the group consisting of age, gender, wheal, size, total IgE, allergen specific IgE, IFNγ, IL-10, IL-13, IL-5 and IL-9; and b) correlating the measurement with allergic status.

In a preferred embodiment, a plurality of variables is measured. In one embodiment, the plurality comprises at least 2 variables. In another embodiment, the plurality comprises at least 3 variables. In a further preferred embodiment, at least four variables are measured.

In a further preferred embodiment, the plurality of variables includes wheal size and total IgE level. In yet another preferred embodiment, the plurality of biomarkers further includes age and IFNγ level.

In one aspect of the invention, the subject is a human subject.

In a preferred embodiment, the allergy is a food allergy. In a particularly preferred embodiment, the food allergy is a peanut allergy.

In one preferred method of the invention, the measurements are correlated with allergic status using a CART analysis tool.

In another aspect of the invention, a software product is provided. The software product comprises: a) code that accesses data relating to at least one variable; and b) code that executes an algorithm that classifies the allergic status of a subject as a function of the data.

In another aspect of the invention, a computer program product is provided. The computer program product comprises computer readable code embodied therein, for execution by a processor of a computing system, device or apparatus, said code configuring the processor to access data relating to at least one variable associated with a subject and selected from the group consisting of: age, gender, wheal size, total IgE, allergen specific IgE, IFNγ, IL-10, IL-13, IL-5 and IL-9; and classifying the allergic status of the subject as a function of the data.

In a preferred embodiment, the processor is configured to classify allergic status as a function of data relating to a plurality of variables. In a further preferred embodiment, at least three variables are analyzed.

In particularly preferred embodiments, methods of diagnosing a true peanut allergy are provided. One method comprises a) performing a skin prick test and measuring the size of the wheal, b) measuring the amount of total IgE in a serum sample, and c) comparing the values obtained to predetermined values wherein a skin test wheal of >4.50 mm and a total IgE level of 95.50 units is indicative of a true peanut allergy and a total IgE level ≦95.50 units indicates a non-true peanut allergy.

Another method of diagnosing a true peanut allergy involves the steps of a) performing a skin prick test and measuring the size of the wheal, b) if the wheal is <4.50 mm, determining if the subject is ≦21.00 years old, wherein there is a low probability that a subject >21.00 years old has a true peanut allergy.

Yet another method of diagnosing a true peanut allergy involves the steps of a) performing a skin prick test and measuring the size of the wheal, b) if the wheal is <4.50 mm, determining if the subject is <21.00 years old, c) measuring total IgE in a serum sample from subjects <21 years old, wherein the probability of true peanut allergy in subjects having a total IgE of >118.50 units is low.

Another method of diagnosing a true peanut allergy involves a decision tree based on the following steps a) performing a skin prick test and measuring the size of the wheal, b) if the wheal is less than <4.50 mm, determining if the subject is <21 years old, c) measuring total IgE in a serum sample from subjects <21 years old, d) for subjects having a total IgE of <118.50 units, measuring IFNγ from a culture of PBMC from the subject exposed to peanut, wherein an IFNγ level >1138.04 pg/ml is indicative of a non-true peanut allergy.

In another aspect of the invention, a kit for the diagnosis of allergy is provided. The kit comprises assay reagents for measuring specified variables and decision tree software for analysis the data and providing a diagnostic prediction.

In a preferred embodiment, the kit predicts the likelihood of a true peanut allergy and reagents are provided for the assay of total IgE in serum.

In another embodiment, the kit further includes reagents for the measurement of IFNγ.

In another preferred embodiment, the kit includes a device for measurement of a SPT wheal.

In one aspect of the invention, a method for diagnosing a food allergy in a subject is provided. The method comprises: a) measuring at least one biomarker selected from the group consisting of age, gender, wheal, size, total IgE, allergen specific IgE, IFNγ, IL-10, IL-13, IL-5 and IL-9; b) determining a biomarker profile and c) correlating the measurement with allergic status.

The method preferably comprises measuring a plurality of the biomarkers, wherein the plurality comprises at least 2 biomarkers.

In a preferred embodiment, the plurality comprises at least 3 biomarkers.

In another preferred embodiment, the plurality of variables includes wheal size and total IgE level.

In a further preferred embodiment the plurality of biomarkers also includes age and IFNγ level.

In preferred embodiments the subject is a human subject and the allergy is a food allergy.

In a more preferred embodiment, the food allergy is a peanut allergy.

In another aspect of the invention, the biomarker profile is correlated with allergic status using a Classification and regression (CART) analysis tool.

In another aspect of the invention a computerized analysis tool is used to predict a food allergy. The computerized tool comprises computer readable code for execution by a processor of a computing system, device or apparatus. The code configures the processor to access inputted data relating to at least one biomarker associated with a subject and selected from the group consisting of: age, gender, wheal size, total IgE, allergen specific IgE, IL-10, IL-13, IL-5 and IL-9; and classifying the allergic status of the subject as a function of the data.

The computing system has computer readable code embodied therein, for execution by a processor of a computing system, device or apparatus, for diagnosing an allergy in a subject. The code configures the processor to implement any of the methods described above.

In another aspect of the invention, a kit for the diagnosis of an allergy is provided. The kit comprises assay reagents for measuring specified biomarkers and decision tree software that analyses the data and provides a diagnostic prediction.

In a preferred embodiment, the kit includes reagents for measuring total IgE in serum.

In a further preferred embodiment, the kit comprises reagents for the measurement of IFNγ.

The kit preferably also includes a device for measurement of a SPT wheal.

In a preferred embodiment, the kit is designed to determine if the allergy is true peanut allergy.

In another aspect of the invention, a method of diagnosing food allergy in a test subject that comprises evaluating whether a plurality of measured parameters in a biomarker profile satisfies a value set, wherein satisfying the value set indicates the presence of a food allergy and wherein the plurality of parameters are measurable aspects of a plurality biomarkers listed in Table 1.

The plurality of biomarkers preferably comprises at least two biomarkers.

In another embodiment, the plurality of biomarkers consists of between 3 and 11 biomarkers.

In a preferred embodiment, the plurality of markers comprises at least two of IFNγ, total IgE, age and wheal size.

In another embodiment, at least one biomarker is tested for in a biological sample from the subject. The biological sample preferably comprises serum, blood, saliva or a tissue sample.

In one embodiment, the plurality of biomarkers are assessed at a single time point.

In another embodiment, the plurality of biomarkers are assessed at different points in time.

In a preferred embodiment, the amplitude of the biomarker measurements increases upon exposure to a food allergen indicating an allergy to the food.

In yet another embodiment, the value set described above is determined by inputting control negative and positive samples for biomarkers to be assessed for inclusion in the value set.

The levels of various biomarkers in control positive and control negative samples are preferably analyzed using a data analysis algorithm to determine a decision rule.

In a preferred embodiment, a Classification and Regression Tree (CART) analysis tool is used.

The decision rule preferably classifies subjects as (i) subjects that have a food allergy and (ii) subjects that are non-allergic to the food allergen, with an accuracy of 90% or better. The food allergy is preferably a peanut allergy.

### BRIEF DESCRIPTION OF THE DRAWINGS

These and other features of the invention will become more apparent from the following description in which reference is made to the appended drawings wherein:

FIG. 1 shows a Classification And Regression Tree (CART) decision making tool in accordance with one embodiment of the present invention; and

FIG. 2 shows a computing system.

### DETAILED DESCRIPTION

Diagnostic methods, tests, tools and automated systems that can accurately inform a physician of the likelihood of a food allergy in a subject have been developed. These are based on the measurement of demographic and/or biological variables. A plurality of biomarkers that form the biomarker profile of the subject are evaluated. The subject is likely to have a food allergy when the biomarker profile meets a predetermined value set.

Quantitative data relating to the biomarkers can be used in diagnostic tests to determine if a subject has a food allergy. The data can be analyzed manually or through specifically designed computer software and/or hardware. The tests of the present invention may be used alone or in combination with other diagnostic tests. The terms “variable” and “biomarker” are used herein interchangeably. Age, gender and wheal size are considered as biomarkers in addition to the other biomarkers measured.

The allergic status of known patient groups (e.g. i) true positives (positive skin test and positive reactivity), ii) true negatives (negative skin test, no reactivity), iii) unknown 1 (positive skin test, no clinical reaction), iv) unknown 2 (positive skin test with no exposure to allergen) can be input to determine which variables or biomarkers or combination of variables are most indicative of allergic status.

Each of the variables listed in Table 1 below may be useful in determining the allergic status of a human subject when in the context of a compatible history. However, while individual variables provide important diagnostic information, it has been found that the accuracy of the diagnosis increases when a combination of variables are used. The analysis of a plurality of variables increases the specificity and sensitivity of the test.

TABLE 1
VARIABLE

Age
Medium condition
Gender
IFNγ
IL-10
IL-13
IL-5
IL-9
Allergen specific IgE
Total IgE
SPT wheal size

The data on the variables may be input into a Classification And Regression Tree (CART) analysis. CART analysis has been used in other applications. The CART program uses a series of “yes/no” questions to construct a decision tree which displays the relationships between important variables in a tree-structured data analysis. The decision tree identifies variables that are most important for predicting an outcome and it shows the relationships among those variables and the outcome. The variables are input into a processor for analysis. It is apparent to one skilled in the art that other types of data analysis could also be used to evaluate the usefulness of certain variables in predicting allergy.

Data reported herein relating to the variables of age, gender, wheal size, total IgE, allergen specific IgE, IFNγ, IL-10, IL-13, IL-5 and IL-9, were input into a computing system and CART generated decision trees to classify a subject as having a true allergy or a true non-allergy. Among the many decision trees generated by CART, a few had excellent sensitivity and specificity to distinguish between allergy and non-allergy.

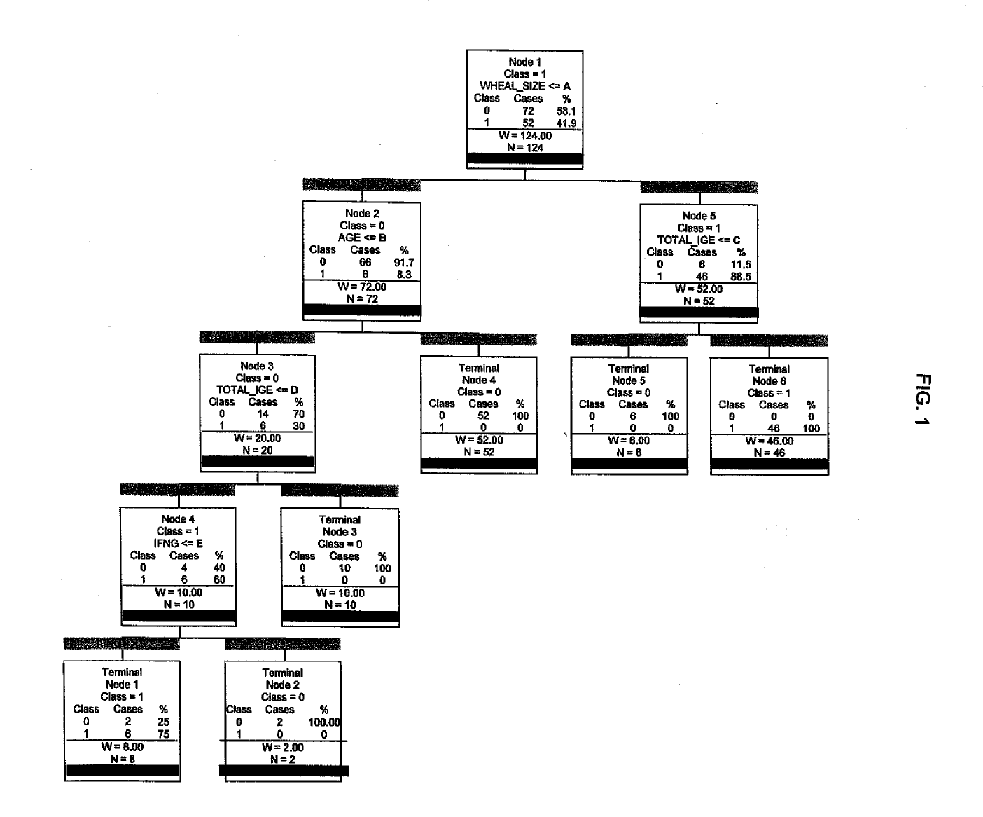

An exemplary decision tree is shown in FIG. 1. For peanut allergy, four variables were found to be particularly useful for predicting reactivity to peanuts. These were IFNγ, total IgE, age and wheal size.

The specifics of the decision trees, such as the branching forks, depend on the cut-off values used. The cut-off values depend on the type of measurement used to generate the data and the differences between control allergic and non-allergic samples.

Variables such as age and gender are readily determined. SPT wheal size is determined using standard measuring techniques.

Antibodies, such as IgE, can be detected in a sample of a bodily fluid such as blood, serum, plasma, saliva, and pleural effusions. A preferred method for the detection of total IgE and/or allergen-specific IgE is an immunoassay. Preferred types of immunoassays include, but are not limited to ELISA, RIA, FIA, agglutination and precipitation. For the methods of the present invention, an ELISA on a serum sample is preferred although other types of immunoassays may also be used to generate the data.

To detect cytokines, such as IFNγ, IL-10, IL-13, IL-5 and IL-9, it is preferable to compare a baseline measurement with a measurement after exposure to an allergen. One way to do this is to isolate peripheral blood mononuclear cells (PBMC) from a patient sample. The PBMC are cultured in the presence and absence of an allergen. Supernatants from the cultures are analyzed to determine the levels of a panel of cytokines relevant to allergy. An immunoassay, such as an ELISA, is typically used to generate the data.

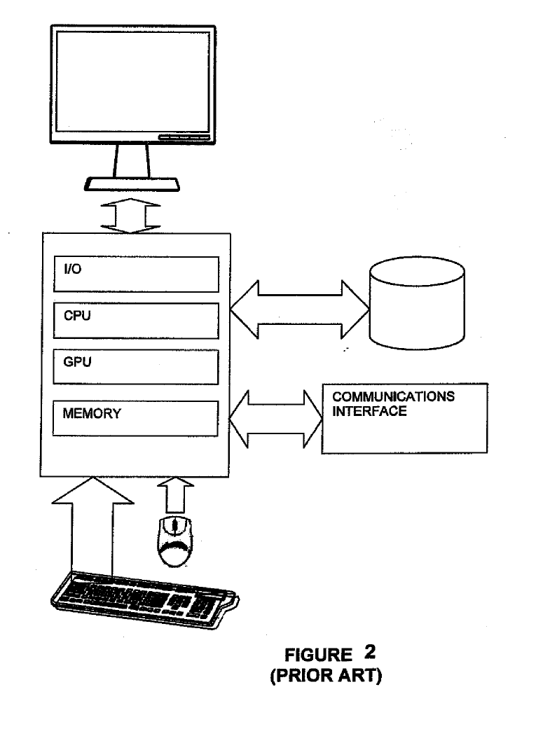

Aspects of the methods of the present invention may be implemented on any suitable computer or microprocessor-based system. An exemplary computer system in respect of which aspects of the present invention may be implemented, is presented as a block diagram in FIG. 2. The exemplary computer system includes a display, input devices in the form of a keyboard and pointing device, computer and external devices. While the pointing device is depicted as a mouse, it will be appreciated that other types of pointing device may also be used.

The computer may contain one or more processors or microprocessors, such as a central processing unit (CPU). The CPU performs arithmetic calculations and control functions to execute software for analysis of variables and relationships between variables and allergy. The software is stored in an internal memory, preferably random access memory (RAM) and/or read only memory (ROM), and possibly additional memory. The additional memory may include, for example, mass memory storage, hard disk drives, optical disk drives (including CD and DVD drives), magnetic disk drives, magnetic tape drives (including LTO, DLT, DAT and DCC), flash drives, program cartridges and cartridge interfaces, removable memory chips such as EPROM or PROM, or similar storage media as known in the art. This additional memory may be physically internal to the computer, or external as shown in FIG. 2.

The computer system may also include other similar means for allowing computer programs or other instructions to be loaded. Such means can include, for example, a communications interface which allows software and data to be transferred between the computer system and external systems and networks. Examples of a communications interface include a modem, a network interface such as an Ethernet card, a wireless communication interface, or a serial or parallel communications port. Software and data transferred via communications interface are in the form of signals which can be electronic, electromagnetic, optical or other signals capable of being received by communications interface. Multiple interfaces, of course, can be provided on a single computer system.

Input and output to and from the computer is administered by the input/output (I/O) interface. This I/O interface administers control of the display, keyboard, external devices and other such components of the computer system. The computer also includes a graphical processing unit (GPU). The system illustrated in FIG. 2 represents a generic computing system and is not meant to limit the claims.

The present invention provides a diagnostic system to correctly predict reactivity to a food, particularly peanut. This is an extremely useful clinical tool which can define the allergic status of children sensitized to peanut, but of unknown clinical reactivity because of peanut avoidance. It introduces an element of certainty which will allow physicians to better identify those for peanut challenge, hence maximizing safety. It is also useful to identify those who have outgrown peanut allergy.

The above disclosure generally describes the present invention. It is believed that one of ordinary skill in the art can, using the preceding description, make and use the compositions and practice the methods of the present invention. A more complete understanding can be obtained by reference to the following specific examples. These examples are described solely to illustrate preferred embodiments of the present invention and are not intended to limit the scope of the invention. Changes in form and substitution of equivalents are contemplated as circumstances may suggest or render expedient. Other generic configurations will be apparent to one skilled in the art. All reference documents referred to herein are hereby incorporated by reference.

### EXAMPLES

Although specific terms have been used in these examples, such terms are intended in a descriptive sense and not for purposes of limitation. Methods of microbiology, molecular biology and chemistry referred to but not explicitly described in the disclosure and these examples are reported in the scientific literature and are well known to those skilled in the art.

### Example 1

### Development of a Diagnostic Tool for Peanut Allergy

125 patients were divided into 4 well-defined groups of peanut allergic and non-allergic individuals. Group 1), patients with a history of a reaction to peanut and a positive skin test to peanut (true positives). Group 2), patients who can eat peanut without any problems yet have a positive skin test to peanut (false positives). Group 3), patients with a positive skin test who have never knowingly ingested peanut. Group 4), patients who are not allergic to peanut and do not have a positive skin test.

PBMCs were isolated and cultured in the presence and absence of peanut. A panel of cytokines relevant to allergy were measured at baseline and in the presence of crude peanut extract. Analysis of the supernatants was performed using a Luminex™ multiELISA system.

Using an advanced statistical method of analysis called CART, an acronym for Classification And Regression Trees, an algorithm to predict peanut allergy was developed. CART is a statistical procedure based on the methodology of binary recursive partitioning. This procedure was introduced by Breimen, Friedman, Olshen and Stone in 1984.

When twelve predictor variables (condition of medium, patient age, patient gender, IFNγ, IL-10, IL-13, IL-5, IL-9, peanut specific IgE on ImmunoCap, SPT wheal size, and total IgE were entered into a CART analysis for patients with a known reactive status to peanut, four variables were identified in the decision tree as being important in terms of predicting reactivity to peanuts. A schematic of the CART analysis performed is provided in FIG. 1. Note that while each variable has a specific value, this value was replaced with a letter in order to keep those involved in the peanut challenges fully blind to the algorithm-predicted outcome. In the schema, Class signifies clinical reactivity (“true peanut allergy=class 1” and “true non-peanut allergy=class 0). Particular attention should be paid to Terminal Nodes; note that there are 5 such nodes in the tree where class predictions are absolute, i.e. 0.0 and 100.0%. The implication, and a finding of central importance, is that the decision tree correctly identified all reactive cases in the tree structure. As with any diagnostic tool, it is important that no case of true peanut allergy be missed. The tree made two errors by misclassifying two non-reactive cases as being reactive. However, it is better to have a false positive than a false negative.

### Example 2

### Validation of the Diagnostic Algorithm

The objective of the second phase of this study is the validation of the diagnostic algorithm. A group of 30 patients of unknown clinical reactivity (Group 3 above) will be studied. 14 patients are enrolled however, these patients will need to be fully re-evaluated, including analysis of PBMC, immediately prior to the oral challenge. The 30 patients will be fed peanut in a double blind placebo controlled fashion using peanut flour according to a published protocol. The tree rules will be applied and a prediction made on clinical reactivity to peanut for each patient. Results will be correlated with their ability to tolerate peanut on feeding. This information can be used to refine and validate a diagnostic algorithm to better predict who is truly allergic to peanut.

Using the foregoing specification, the invention may be implemented as a machine, process or article of manufacture by using standard programming and/or engineering techniques to produce programming software, firmware, hardware or any combination thereof.

Any resulting program(s), having computer-readable program code, may be embodied within one or more computer-usable media such as memory devices or transmitting devices, thereby making a computer program product or article of manufacture according to the invention. As such, the terms “software” and “application” as used herein are intended to encompass a computer program existent (permanently, temporarily, or transitorily) on any computer-usable medium such as on any memory device or in any transmitting device.

Examples of memory devices include, hard disk drives, diskettes, optical disks, magnetic tape, semiconductor memories such as FLASH, RAM, ROM, PROMS, and the like. Examples of networks include, but are not limited to, the Internet, intranets, telephone/modem-based network communication, hard-wired/cabled communication network, cellular communication, radio wave communication, satellite communication, and other stationary or mobile network systems/communication links.

A machine embodying the invention may involve one or more processing systems including, for example, CPU, memory/storage devices, communication links, communication/transmitting devices, servers, I/O devices, or any subcomponents or individual parts of one or more processing systems, including software, firmware, hardware, or any combination or subcombination thereof, which embody the invention as set forth in the claims.

Using the description provided herein, those skilled in the art will be readily able to combine software created as described with appropriate general purpose or special purpose computer hardware to create a computer system and/or computer subcomponents embodying the invention, and to create a computer system and/or computer subcomponents for carrying out the method of the invention.

One or more currently preferred embodiments have been described by way of example. It will be apparent to persons skilled in the art that a number of variations and modifications can be made without departing from the scope of the invention as defined in the claims.

What is claimed:

1. A method for diagnosing a food allergy in a subject, said method comprising:
a) measuring at least one biomarker selected from the group consisting of age, gender, wheal, size, total IgE, allergen specific IgE, IFNγ, IL-10, IL-13, IL-5 and IL-9;
b) determining a biomarker profile and
c) correlating the measurement with allergic status.

2. The method of claim 1, comprising measuring a plurality of said biomarkers.

3. The method of claim 2, wherein the plurality comprises at least 2 biomarkers.

4. The method of claim 3, wherein the plurality comprises at least 3 biomarkers.

5. The method of claim 2, wherein the plurality of variables includes wheal size and total IgE level.

6. The method of claim 5, wherein the plurality of biomarkers further includes age and IFNγ level.

7. The method of claim 1, wherein the subject is a human subject.

8. The method of claim 1, wherein the allergy is a food allergy.

9. The method of claim 8, wherein the food allergy is a peanut allergy.

10. The method of claim 1, wherein the measurements are correlated with allergic status using a CART analysis tool.

11. A computer program product having computer readable code embodied therein, for execution by a processor of a computing system, device or apparatus, said code configuring the processor to access data relating to at least one biomarker associated with a subject and selected from the group consisting of: age, gender, wheal size, total IgE, allergen specific IgE, IL-10, IL-13, IL-5 and IL-9; and classifying the allergic status of the subject as a function of the data.

12. A computer program product having computer readable code embodied therein, for execution by a processor of a computing system, device or apparatus, for diagnosing an allergy in a subject said code configuring the processor to implement the method of any one of claims 1 to 10.

13. A kit for the diagnosis of allergy comprising assay reagents for measuring specified biomarkers and decision tree software that analyses the data and provides a diagnostic prediction.

14. A kit according to claim 13 wherein the allergy is true peanut allergy and reagents for measuring total IgE in serum.

15. A kit according to claim 14 further comprising reagents for the measurement of IFNγ.

16. A kit according to claim 14 further comprising a device for measurement of a SPT wheal.

17. A method of diagnosing food allergy in a test subject, said method comprising evaluating whether a plurality of parameters in a biomarker profile satisfies a value set, wherein satisfying the value set indicates the presence of a food allergy and wherein the plurality of parameters are measurable aspects of a plurality biomarkers listed in Table 1.

18. The method of claim 17, wherein the plurality of biomarkers comprises at least two biomarkers.

19. The method of claim 17, wherein the plurality of biomarkers consists of between 3 and 11 biomarkers.

20. The method of claim 17, wherein the plurality of markers comprises at least two of IFNγ, total IgE, age and wheal size.

21. The method of claim 17, wherein at least one biomarker is tested for in a biological sample from said subject.

22. The method of claim 21, wherein the biological sample comprises serum, blood, saliva or a tissue sample.

23. The method of claim 17, wherein the plurality of biomarkers are assessed at a single time point.

24. The method of claim 17, wherein the plurality of biomarkers are assessed at different points in time.

25. The method of claim 24, wherein the amplitude of the biomarkers increases upon exposure to a food allergen.

26. The method of claim 17, wherein the value set is determined by inputting control negative and positive samples for biomarkers to be assessed for inclusion in the value set.

27. The method of claim 26, wherein the levels of various biomarkers in control positive and control negative samples are analyzed using a data analysis algorithm Classification and Regression Tree (CART) analysis tool.

28. The method of claim 27, wherein the decision rule classifies subjects as (i) subjects that have a food allergy and (ii) subjects that are non-allergic to the food allergen, with an accuracy of 90% or better.

29. The method of claim 27, wherein the food allergy is a peanut allergy.
